(19) United States
(12) Plant Patent Application Publication
(10) Pub. No.: US 2005/0262601 P1
(43) Pub. Date: Nov. 24, 2005

(54) Miniature rose plant 'PACfourteen'

(50) ROSA HYBRIDA; SUPER MINIATURE ROSE

(76) Inventor: Dan Jauchen, Santa Barbara, CA (US)

Correspondence Address: Dan Jauchen;905 South Patterson Avenue, P.O. Box 6354, Santa Barbara, CA, 93111, US

(21) Appl. No.: 10/851,296

(22) Filed: May 24, 2004

(51) Int. Cl. A01H 5/00

(52) U.S. Cl. PLT/128

(57) ABSTRACT

A new miniature rose plant which has abundant, pink colored flowers and attractive foliage. The variety successfully propagates from softwood cuttings and is suitable for year round production in commercial glass houses as a flowering pot plant. This new and distinct variety has shown to be uniform and stable in the resulting generations from asexual propagation.

### BACKGROUND OF THE INVENTION

The present invention constitutes a new and distinct variety of miniature rose plant, which was developed by artificially pollinating an unnamed seedling(not patent in the US) with an unnamed seedling (not patent in the US). The two parents were crossed in the summer of 2001 and the resulting seed was sown in December 2001, in a controlled glasshouse environment. Out of these seedlings one seedling was selected, as the new variety and named ‘PACfourteen’. The new rose may be distinguished from its seed parent, an unnamed seedling, by the following combination of characteristics:
1. The unnamed seedling has a breeding background in unnamed seedlings.
2. ‘PACfourteen’ has medium double flowers, while the unnamed seedling has big single flowers.
3. ‘PACfourteen’ has pink colored petals, while the unnamed seedling has white petals.

The new variety may distinguished from its pollen parent, an unnamed seedling created by the same inventor, by the following combination of characteristics:
1. The unnamed seedling has a breeding background in unnamed seedlings.
2. ‘PACfourteen’ has biggrt flowers and foliage as compared to the unnamed seedling.
3. ‘PACfourteen’ has pink colored petals, while the unnamed seedling has dark orange petals.

### BRIEF SUMMARY OF THE INVENTION

Initial asexual reproduction of ‘PACfourteen’ by cuttings was first done in Santa Barbara, Calif., USA. The reproduction was conducted in controlled greenhouse environments.

Have here proven that the foregoing and all after characteristics and distinctions to come true to form and are established in succeeding propagations. ‘PACfourteen’ is a low and compact miniature rose with good vigor. The shelflife for the entire plant and the single flush, has been tested to be good.

The objective of the hybridization of this rose variety for commercial greenhouse culture was to create a new and distinct variety with:

1. Uniform and abundant flowers with good keepability;

2. Attractive long lasting foliage and compact growth,

3. Year round flowering under glasshouse conditions;

4. Suitability for production from softwood cuttings in pots.;

5. Durable flowers and foliage which make the variety suitable for distribution in the floral industry.

This combination of qualities was not present in previously available commercial cultivars of this type and distinguish ‘PACfourteen’ from all other varieties of which we are aware.

The seeds from hybridization were planted in a controlled environment and evaluations were conducted on the resulting plants. ‘PACfourteen’ was selected by, Dan Jauchen, in his development program in Santa Barbara, Calif., USA.

### BRIEF DESCRIPTION OF THE DRAWINGS

The accompanying color illustrations show as true as is reasonably to obtain in color photographs of this type, the typical characteristics of the buds, flowers, leaves, stems of ‘PACfourteen’. Specifically illustrated in:

### Photo Sheet #1

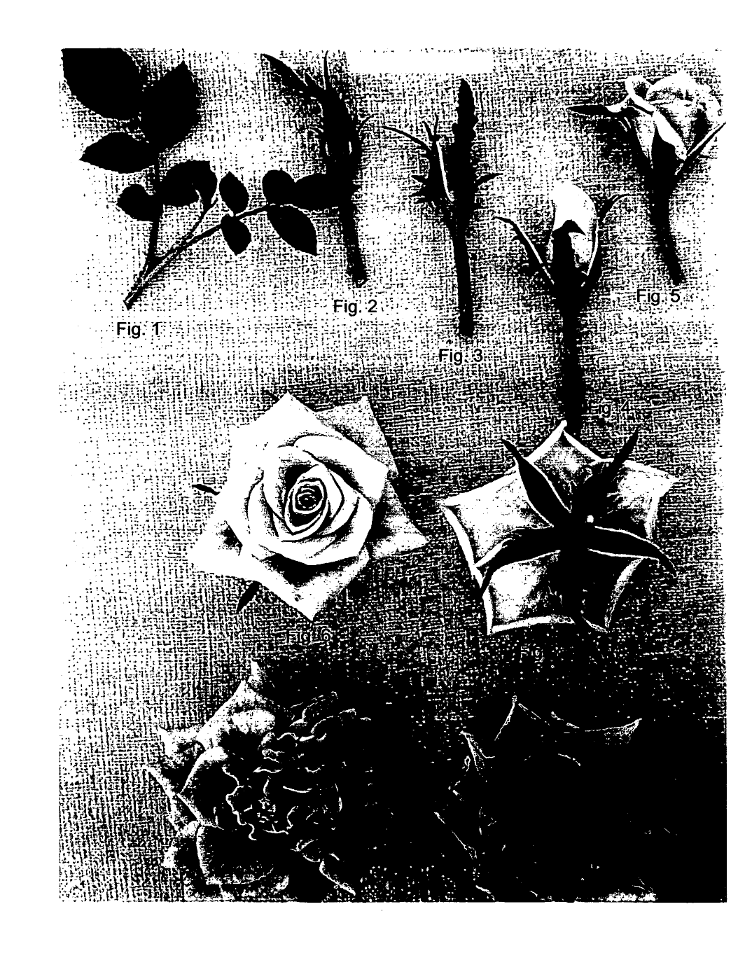

FIG. 1 Young shoot.

FIG. 2 Bud before opening the sepals.

FIG. 3 Bud at the stage of opening the sepals.

FIG. 4 Bud at the stage of opening the petals.

FIG. 5 Flower during course of opening.

FIG. 6 Open flower—plan view—obverse.

FIG. 7 Open flower—plan view—reverse.

FIG. 8 Fully open flower—plan view—obverse.

FIG. 9 Fully open flower—plan view—reverse.

### Photo Sheet #2

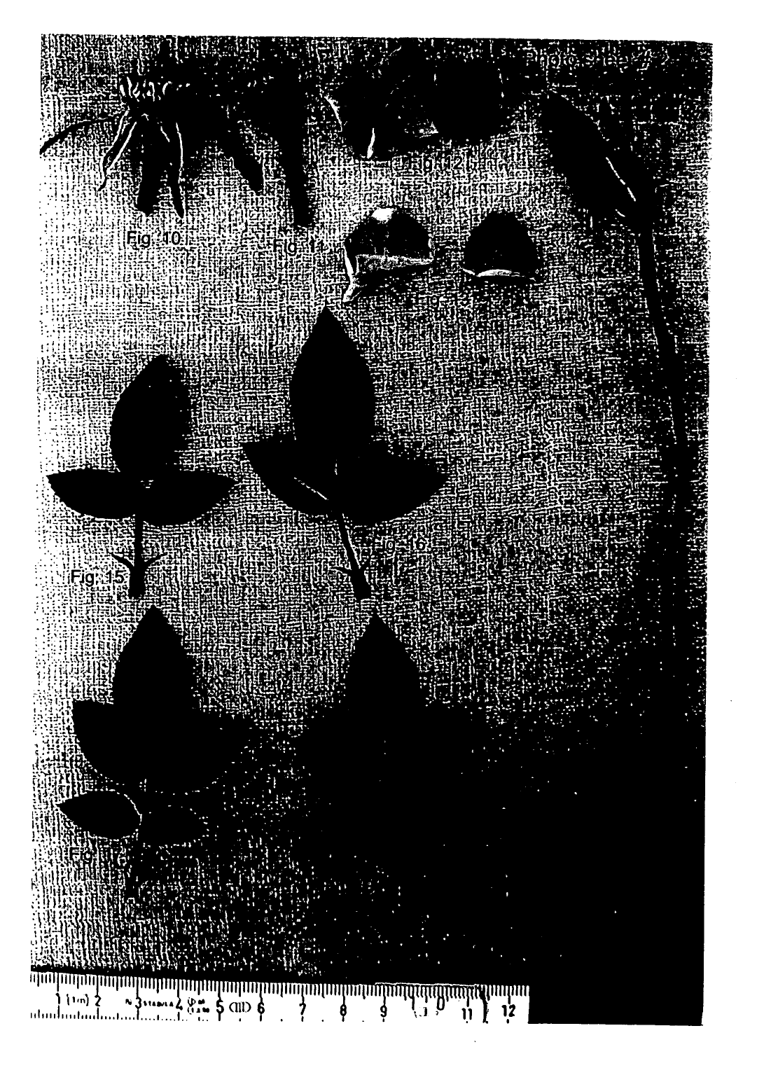

FIG. 10 Receptacle showing stamens and pistils.

FIG. 11 Receptacle showing pistils (stamens and sepals removed).

FIG. 12 Flower petals, detached—inner surface.

FIG. 13 Flower petals, detached—outer surface.

FIG. 14 Bare stem exhibiting thorns and flower attachment.

FIG. 15 Three leaflets upper side.

FIG. 16 Three leaflets reverse side.

FIG. 17 Five leaflets upper side.

FIG. 18 Five leaflets reverse side.

### DETAILED BOTANICAL DESCRIPTION OF THE VARIETY

The following is a detailed description of the Miniature Rose: Rosa hybrid ‘PACfourteen’.

The following observations, measurements, values and comparisons describe plants grown in glass houses in Santa Barbara, Calif., USA.

The age of the observed plants where 11 to 13 weeks after propagation by cuttings, and produced as flowering pot plants in container of 15 centimeter in diameter.

Color references are made using The Royal Horticultural Society (London, England) Colour Chart, 1995, except where common terms of color are used. For a comparison, the nearest existing rose variety is ‘KORpikula’, a rose variety described and illustrated in U.S. Plant Pat. No. 12,583.

Chart 1 details several physical characteristics of ‘PACfourteen’ and ‘KORpikula’.

CHART 1

| | ‘PACfourteen’ | ‘KORpikula’ |
|---|---|---|
| Petal color, Upper | Red Group | Red Group |
| surface | 49A | 49C |
| Petal color, Reverse | Red Group | Red Group |
| surface | 49B | 49C |
| Petal count | 40 | 30-35 |

Parents.—Unnamed seedling. Times. Unnamed seedling.
Classification:
   Botanical.—Rosa hybrid.
   Commercial.—Miniature.
Plant:
   Plant growth.—Moderately vigorous. Grows compact upright to bushy. When grown as 10 cm pot plant, the average height of the plant itself is 18 to 20 cm, and average with is 20 cm. When grown as a 15 cm pot plant, the average height of the plant itself is 22 to 27 cm, and average with is 30 cm. Production time is generally 11 to 13 weeks depending on average temperature, light level, and cultural practices.
Stem:
   Color.—Young wood: Yellow-Green Group 148B. Older wood: Yellow-Green Group 147A.
   Thorns.—Incidence: Low number of thorns Size: 3-4 mm Color: Greyed-Purple Group 185C Shape: Deep concave.
   Surface.—Young wood: Smooth Older wood: Smooth.
   Stem diameter.—2-4 mm
   Internode length.—20-30 mm.
   Numbers of internodes.—7-9.
Plant foliage: Leaves arranged alternately, compound with 3.5 to 7 leaflets per leaf, generally symmetrical, abundant, and flat in aspect. Stipules at petiole base.
   Quantity of leaves.—7 to 9 per lateral branch.
   Leaf size.—Medium 65-70 mm(l). times. 40-45mm(w), for 5 leaflet.
   Petioles.—Color: Yellow-Green Group 147B-C, with intonations of Greyed-Purple Group 183C Margins: with small prickles Greyed-Purple Group 183C Length: 8-10 mm Diameter: about 0.5-1 mm.
   Stipules.—Size: 5-6 mm Surface: Smooth Color Yellow-Green Group 147B-C, Margins: Serrated single.
   Rachis.—Color: Yellow-Green Group 147B-C, with intonations of Greyed-Purple Group 183C Margins: With small prickles, Greyed-Purple Group 183C. Length: 20 to 25 mm.
   Leaflets.—Edge: Serrated Serration: Single Shape: Ovate with acute apex and obtuse base Texture: Smooth Appearance: Dull Size: length: 30 to 35 mm. Width: 10 to 18 mm Color Young foliage: Upper surface: Yellow-Green Group 146A With intonations of Greyed-Red 178B Lower surface: Yellow-Green Group 146C, With intonations of Greyed-Red 178B Color: Mature foliage: Upper surface: Yellow-Green Group 147A Lower surface: Greyed-Green Group 191B With Intonations of Greyed-Purple Group 178B.
Inflorescense:
   Blooming habit.—Recurrent.
   Number of flowers.—Generally 1 bud per flowering stem.
   Peduncle.—Color: Yellow-Green Group 146B, With stipitate glands, Greyed-Red Group 178B Texture: Smooth Length: 25-30 mm Strength: Upright.
   Receptacle.—Surface: Smooth, glabrous. Shape: Funnel-shaped Size: h: 5-6 mm w:6-7 mm Color Yellow-Green Group 146B.
   Sepals.—-Quantity: 5 Shape: Narrowly Ovate w. acute tip Texture: Leathery Margin: Foliaceous appendages on 3 of the five sepals Appearance: Dull Color: Upper surface: Yellow-Green Group 147A Reverse surface: Greyed-Green Group 189A.
   Buds.—Size: 14-17 mm (h) 12-15 mm (w)upon opening. Shape: Pointed Color: at ¼ opening, Red Group 49A.
   Flower.—
   Duration.—As a pot plant, flowers last form 14 to 18 days. Fragrance. None. Size: 55-65 mm in diameter. Form: Shape of flower when viewed from the side. Up on opening: Cupped to pointed Open flower Flat Color: Petals, upon opening. Upper surface: Red Group 49A, With intonations of Red-Purple Group 58B on outer petals. Orange-Red Group 33A on inner petals. Reverse surface: Red Group 49B, With intonations of Red-Purple Group 58B on outer petals Orange-Red Group 33A on inner petals. Petals after opening: Upper surface: Red Group 49A, With intonations of Red-Purple Group 58B on outer petals. Orange-Red Group 33A on inner petals. Reverse surface: Red Group 49B, With intonations of Red-Purple Group 58B on outer petals Orange-Red Group 33A on inner petals. Basal Petals spots: Size: 2-3 mm Color: White Group 155D General tonality: On Open flower: Third day: Red Group 49A Afterwards: Red Group 49B With intonations of Orange-Red Group 33A.
   Petals.—
   Petal reflex.—Outermost petals reflex backwards at opening . Fully open all petals reflex backwards.
   Texture.—Smooth.
   Petal edge.—Uniform.
   Petal count.—Approximately 40 on the average per flower.
   Petal size.—Length 25-30 mm Width: 15-30 mm.
   Shape.—Outer petals: Round — ovate Inner petals: Ovate.
   Reproductive organs.—
   Stamen number.—Approximately 40 on average per flower.
   Pollen. Color.—Yellow-Orange Group 22B, Abundance: high.
   Anthers.—Size: 1-2 mm Color: Yellow-White Group 167B, Shape: Oblong.
   Filaments.—Size: 3-5 mm Color: Greyed-Orange Group 158D.
   Pistils number.—Approximately 40-50 on average per flower.
   Stigmas.—Location: Lower in location to anthers Color: White Group 155B.
   Styles.—Color: Red Group 53B. Length: 1 to 2 mm.
Development:
   Vegetation.—Dense.
   Blooming.—Abundant.
   Aptitude to bear fruit.—Poor.
   Resistance to diseases.—Above average resistance to mildew and Botrytis under normal growing conditions in Santa Barbara, Calif., USA. Hips/seeds has not been observed due to that the plant has never been grown to the stage of seed development, due to the fact, that the variety is developed for use as a flowering pot plant only.
   Winter hardiness & Drought/heat tolerance.—Due to the fact, that this variety is a potted flowering plant, developed indoor use only, the plant is not tested for winter hardiness or drought/heat tolerance.

1. A new and distinct variety of rose plant of the miniature class, substantially as herein illustrated and described as a distinct and novel rose variety due to its abundant pink flowers, attractive long lasting foliage, vigorous and compact growth, year round flowering under glasshouse conditions, suitability for production from softwood cuttings in pots, and durable flowers and foliage which make the variety suitable for distribution in the floral industry.
